(19) United States
(12) Patent Application Publication
(10) Pub. No.: US 2024/0233125 A9
(43) Pub. Date: Jul. 11, 2024

(54) METHOD OF EXTRACTING GENE CANDIDATE, METHOD OF UTILIZING GENE CANDIDATE, AND COMPUTER-READABLE MEDIUM

(71) Applicants: Evident Corporation, Kamiina-gun (JP); FUKUSHIMA MEDICAL UNIVERSITY, Fukushima-shi (JP)

(72) Inventors: Kosuke TAKAGI, Kamiina-gun (JP); Motoki TAKAGI, Fukushima-shi (JP); Kazuhito GODA, Kamiina-gun (JP)

(73) Assignees: Evident Corporation, Kamiina-gun (JP); FUKUSHIMA MEDICAL UNIVERSITY, Fukushima-shi (JP)

(21) Appl. No.: 18/379,834

(22) Filed: Oct. 13, 2023

(30) Foreign Application Priority Data
Oct. 21, 2022 (JP) 2022-168731

(51) Int. Cl. G06T 7/00; G16B 25/10; G16B 40/20; G16H 20/10; G16H 50/20

(52) U.S. Cl. CPC G06T 7/0016; G16B 25/10; G16B 40/20; G16H 20/10; G16H 50/20; G06T 2207/10056; G06T 2207/30024

(57) ABSTRACT

A microscope image of a cultured cell cluster derived from a cancer specimen of a patient is acquired. A measured value of a gene expression level of the cluster is acquired. Based on the image, a morphological representation identifiably expressing, by a vector quantity of a plurality of dimensions, a morphological difference between a group of cell clusters cultured from the same cancer specimen and a group of cell clusters cultured from another cancer specimen is acquired. The acquired morphological representation is input to a function, which is obtained by fitting the measured value with respect to the morphological representation, to acquire a prediction value of the gene expression level. Prediction accuracy is estimated based on the prediction value and the measured value. Based on the estimated prediction accuracy, a gene related to a morphological change of the cell cluster is extracted as a gene candidate.

### CROSS REFERENCE TO RELATED APPLICATIONS

This application is based upon and claims the benefit of priority from prior Japanese Patent Application No. 2022-168731, filed Oct. 21, 2022, the entire contents of which are incorporated herein by this reference.

### BACKGROUND OF THE INVENTION

### Field of the Invention

The disclosure of the present specification relates to a method of extracting gene candidates related to the cancers of the individual patients by imaging (acquiring images of) cultured samples or cell clusters using the cells derived from patients and analyzing them, a method of utilizing the gene candidates, and a computer-readable medium.

### Description of the Related Art

Recently, attempts to make use of the information unique to patients such as genes for diagnosis or treatment of cancers have been actively made. For example, it is known that mutation of particular genes and expression changes caused along therewith are related to the degrees of malignancy of cancers, effectiveness of anticancer drugs, etc. (for example, so-called cancer-related genes or cancer gene markers). Recently, study that carries out integrated analysis combining the data of, for example, genes related to cancer conditions and clinical information has been actively carried out and has become a field that attracts attention.

Cancers and tumors include uneven and diverse cells. Therefore, cancers and tumor conditions exhibit complex aspects. This is one of major features of tumors. Recently, reproducing cell clusters (three-dimensional structures called organoids), which imitate behavior in living bodies, in wells such as multiwell plates has attracted attention as an effective means for studying these complex tumors. As indicators which feature the complexity of the tumors, gene expressions and medication reaction have been used. Using microscope images is also considered to give important clues for understanding complex behavior in organoid samples.
(Non-Patent Literature 1) “A review on machine learning principles for multi-view biological data integration”, Briefings in Bioinformatics, Volume 19, Issue 2, March 2018, Pages 325-340
(Non-Patent Literature 2) “Multi-omic and multi-view clustering algorithms review and cancer benchmark”, Nucleic Acids Research, Volume 46, Issue 20, 16 Nov. 2018, Pages 10546-10562
(Non-Patent Literature 3) “Integrating spatial gene expression and breast tumour morphology via deep learning”, Nature Biomedical Engineering, Volume 4, Pages 827-834 (2020)
(Non-Patent Literature 4) “Pheno-seq—linking visual features and gene expression in 3D cell culture systems”, Scientific Reports, Volume 9, Article number 12367 (2019)

### SUMMARY OF THE INVENTION

A method according to an aspect of the present invention is a method of extracting a gene candidate related to a feature of a cancer of an individual patient, the method including: (a) acquiring an image of a cultured cell cluster derived from a cancer specimen of the patient by using a microscope; (b) measuring a gene expression level of the cancer specimen or the cell cluster cultured from the cancer specimen used in the (a); (c) acquiring a morphological representation identifiably expressing, by a vector quantity of a plurality of dimensions, a morphological difference between a group of a cell cluster cultured from the same cancer specimen and a group of a cell cluster cultured from another cancer specimen based on the image acquired in the (a); (d) fitting a function so that the gene expression level measured in the (b) is output with respect to input of the morphological representation acquired in the (c); (e) estimating prediction accuracy of the gene expression level by comparing a prediction value of the gene expression level, which is output of the function subjected to fitting in the (d), with a measured value of the gene expression level measured in the (b); and (f) selecting a gene related to a morphological change of the cell cluster based on the prediction accuracy estimated in the (e) and extracting the gene candidate based on the selected gene.

A method according to another aspect of the present invention is a method of utilizing a gene candidate extracted by using the method of extracting the gene candidate according to the above described aspect, the method including a procedure of supporting classification or diagnosis of a cancer of a patient or predicting an effect of medication with respect to the patient by using the prediction value of the gene expression level of the gene candidate.

A non-transitory computer-readable medium according to an aspect of the present invention stores a program that causes a computer to execute: (a) acquiring an image of a cultured cell cluster derived from a cancer specimen of a patient by using a microscope; (b) measuring a gene expression level of the cancer specimen or the cell cluster cultured from the cancer specimen used in the (a); (c) acquiring a morphological representation identifiably expressing, by a vector quantity of a plurality of dimensions, a morphological difference between a group of a cell cluster cultured from the same cancer specimen and a group of a cell cluster cultured from another cancer specimen based on the image acquired in the (a); (d) fitting a function so that the gene expression level measured in the (b) is output with respect to input of the morphological representation acquired in the (c); (e) estimating prediction accuracy of the gene expression level by comparing a prediction value of the gene expression level, which is output of the function subjected to fitting in the (d), with a measured value of the gene expression level measured in the (b); and (f) selecting a gene related to a morphological change of the cell cluster based on the prediction accuracy estimated in the (e) and extracting the gene candidate related to a feature of the cancer of the patient based on the selected gene.

### BRIEF DESCRIPTION OF THE DRAWINGS

The present invention will be more apparent from the following detailed description when the accompanying drawings are referenced.

FIG. 1 is a diagram illustrating a flow of a series of processes using deep neural networks (DNN);

FIG. 2A to FIG. 2C are schematic diagrams illustrating microscope images of F-PDO;

FIG. 3A to FIG. 3C are schematic diagrams illustrating different microscope images of F-PDO;

FIG. 4A to FIG. 4C are schematic diagrams illustrating further different microscope images of F-PDO;

FIG. 5A to FIG. 5C are schematic diagrams illustrating further different microscope images of F-PDO;

FIG. 6 is a diagram illustrating a state of generating image patches from a microscope image;

FIG. 7 is a diagram illustrating a layer configuration example of a first model;

FIG. 8 is a diagram visualizing image representations, which are obtained by the first model, by using a dimension reduction technique;

FIG. 9 is a diagram visualizing patterns of the image representations;

FIG. 10 is a diagram exemplifying color images acquired by color CCD;

FIG. 11A and FIG. 11B are diagrams visualizing, by using a dimension reduction technique, the image representations obtained by the first model and image representations obtained by an autoencoder, respectively;

FIG. 12A and FIG. 12B are diagrams visualizing patterns of the image representations obtained by the first model and patterns of the image representations obtained by the autoencoder, respectively;

FIG. 13 is a diagram illustrating a layer configuration example of a second model;

FIG. 14 is a diagram visualizing gene expression levels measured for each label;

FIG. 15 is a diagram illustrating the relations between prediction accuracy and variance of the gene expression levels of genes;

FIG. 16 is a diagram for describing a method of extracting candidate genes;

FIG. 17 is a diagram illustrating a layer configuration example of a third model;

FIG. 18 is a diagram visualizing the drug response measured for each label;

FIG. 19 is a diagram illustrating prediction accuracy of drug responsiveness;

FIG. 20 is a diagram illustrating an example of a system configuration;

FIG. 21 is a diagram illustrating another example of the system configuration;

FIG. 22 is a diagram exemplifying a hardware configuration of a computer for implementing a system; and

FIG. 23 is a flow chart of a process of extracting and utilizing gene candidates.

### DESCRIPTION OF THE EMBODIMENTS

Organoids reflect the features of individual tumors. On the other hand, it is difficult to specify the types of the features. Specifically, the cultured organoids derived from the same cancer patient sometimes exhibit diversity such as morphological differences or size differences. Due to the foregoing, it is difficult to specify the features common to the organoids cultured from the same cancer. Also, it has been difficult to simply quantify morphological features from microscope images of organoids, which have complex morphological features, and extract (find out) common features from the data obtained therefrom.

Therefore, regarding the organoid samples derived from a common tumor having complex and diverse morphological features, a method of finding out the cause and factors featuring a condition of a patient has been desired. Specifically, identifying the genes which cause differences in medication reaction is expected to contribute to, for example, prediction of medication efficacy and development of new medication.

In view of the foregoing circumstances, it is an object of an aspect of the present invention to specify gene candidates related to the features of cancers of the same type by using microscope images of, for example, organoid samples having complex morphological features.

According to a method described in a following embodiment, the gene candidates related to the features of the cancers of the same type can be specified by using microscope images.

Hereinafter, a method of extracting gene candidates related to the features of cancers of individual patients and a method of utilizing the gene candidates will be described. These methods have been invented through study using gene expressions, medication reaction, and a data set of microscope images of lung-cancer-derived organoids of the patient-derived tumor organoid collection of Fukushima Medical University (F-PDO (registered trade name)).

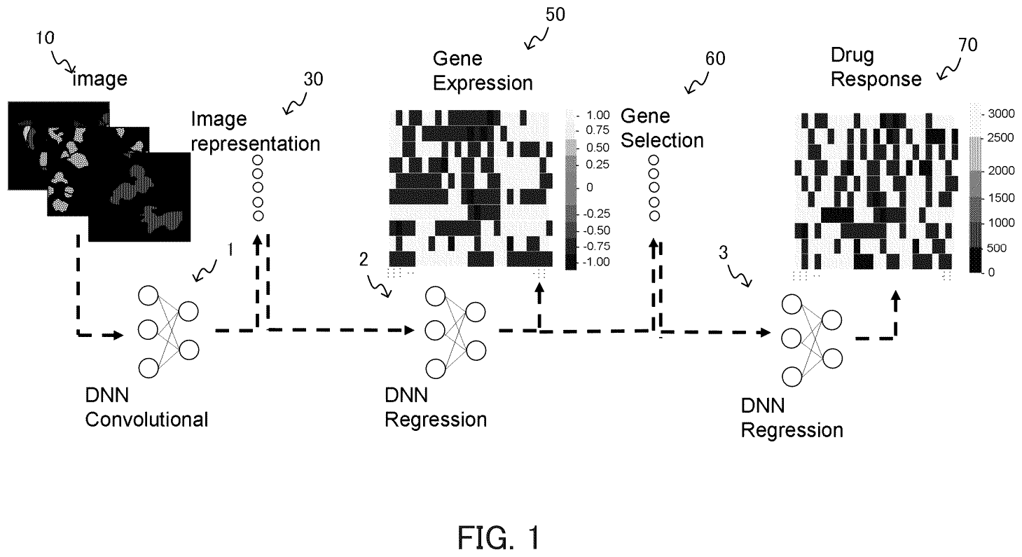

FIG. 1 is a diagram illustrating a flow of a series of processes using deep neural networks (DNN). As illustrated in FIG. 1, the above described method of extracting gene candidates and the method of utilizing the gene candidates are carried out desirably by using three deep neural networks (model 1, model 2, and model 3).

As illustrated in FIG. 1, input data set 10 is sequentially processed stepwise by the deep neural networks. First, the model 1 which is a convolutional neural network (CNN) for images is applied, the model 2 which is a regression model for gene expression level prediction is then applied, and the model 3 which is a regression model for medication reaction prediction is applied. The input data set 10 is input image data set and is, more specifically, a data set of microscope images. In the present study, the data set of the microscope images of above described F-PDO (registered trade name) is used as the input data set 10.

<First Model>

The model 1, which is a first model, generates an image representation 30 (also referred to as an image feature vector) by converting the dimensions of the input data set 10 to a vector of smaller dimensions. The model 1 and the image representation 30 generated by using the model 1 will be described.

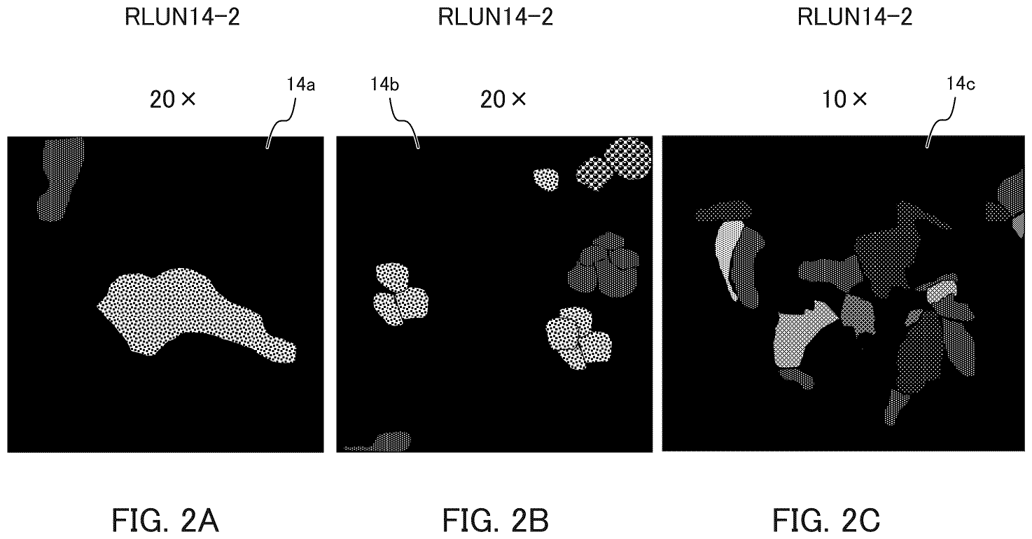

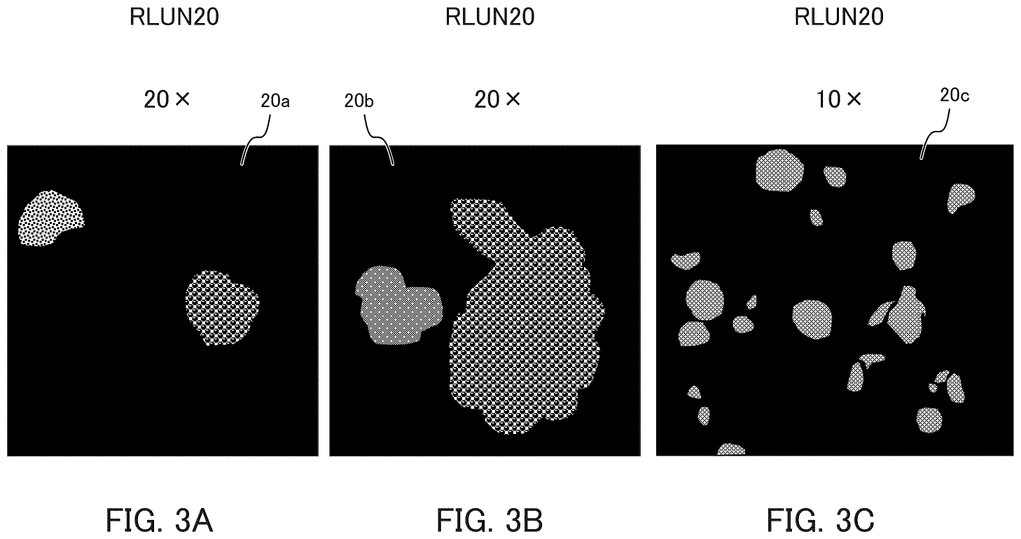

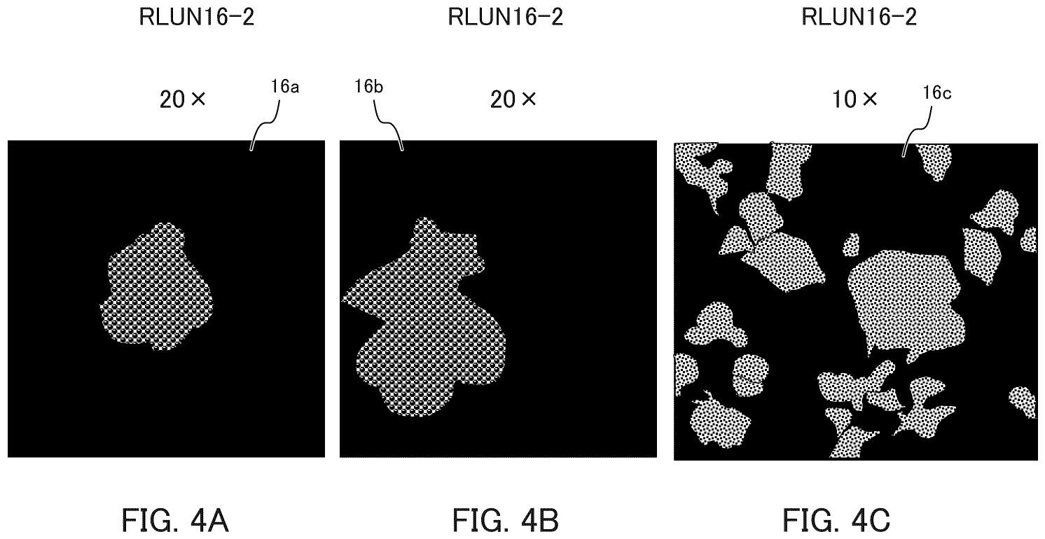

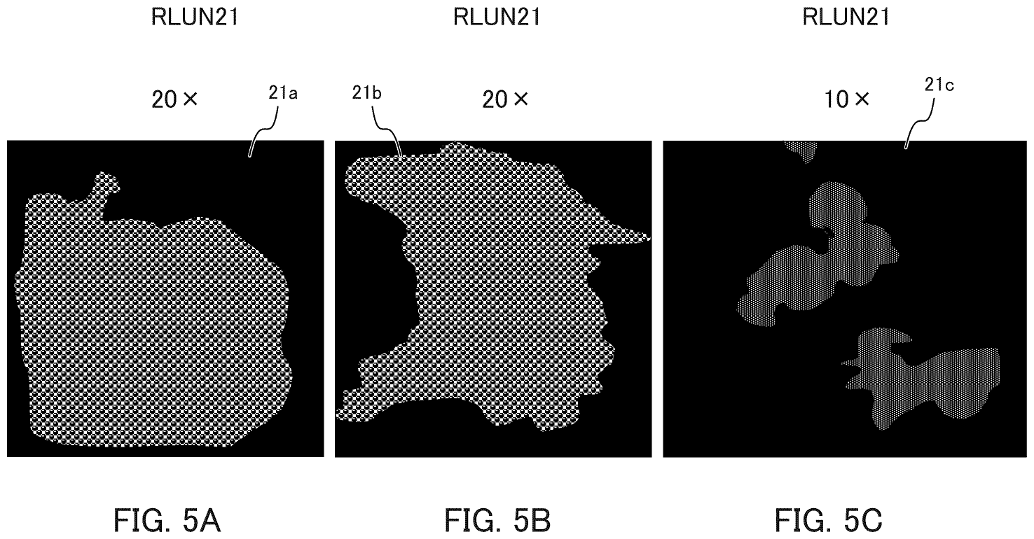

FIG. 2A to FIG. 5C are schematic diagrams illustrating the microscope images of F-PDO. Labels are attached to the microscope images of F-PDO in advance. The labels represent deriving source tumors, in other words, types of cancers and represent a deriving source patient. Each of FIG. 2A to FIG. 2C, FIG. 3A to FIG. 3C, FIG. 4A to FIG. 4C, and FIG. 5A to FIG. 5C illustrates three images to which the same label is attached. In other words, the three images of each of FIG. 2A to FIG. 2C, FIG. 3A to FIG. 3C, FIG. 4A to FIG. 4C, and FIG. 5A to FIG. 5C illustrate the microscope images derived from the same cancer patient.

FIG. 2A and FIG. 2B illustrate grayscale images (image 14a, image 14b) acquired by a microscope using a 20-power phase-contrast objective lens. FIG. 2C is a color image (image 14c) acquired by a microscope equipped with a color CCD camera using a 10-power bright-field objective lens. All of the images are denoted with a label name “RLUN14-2”.

The three images (image 20a, image 20b, image 20c, image 16a, image 16b, image 16c, image 21a, image 21b, and image 21c) of each of FIG. 3A to FIG. 3C, FIG. 4A to FIG. 4C, and FIG. 5A to FIG. 5C are the images acquired by similar settings as the three images of FIG. 2A to FIG. 2C and are denoted with label names “RLUN20”, “RLUN16-2”, and “RLUN21”, correspondingly.

As illustrated from FIG. 2A to FIG. 5C, F-PDO includes non-uniform cells and has complex morphologies.

In the present study, in order to collect learning data of the model 1, 20 grayscale images of 1360×1024 pixels have been captured by using a 20-power phase-contrast objective lens for each of the samples (specimens) to which above described four types of labels are attached. Also, in order to confirm that the model 1 effectively functions also for the images which have been captured with different settings, four color images of 1920×1440 pixels are captured by using a 10-power bright-field objective lens and a color CCD for the samples to which labels of 25 types including the above described four types are attached.

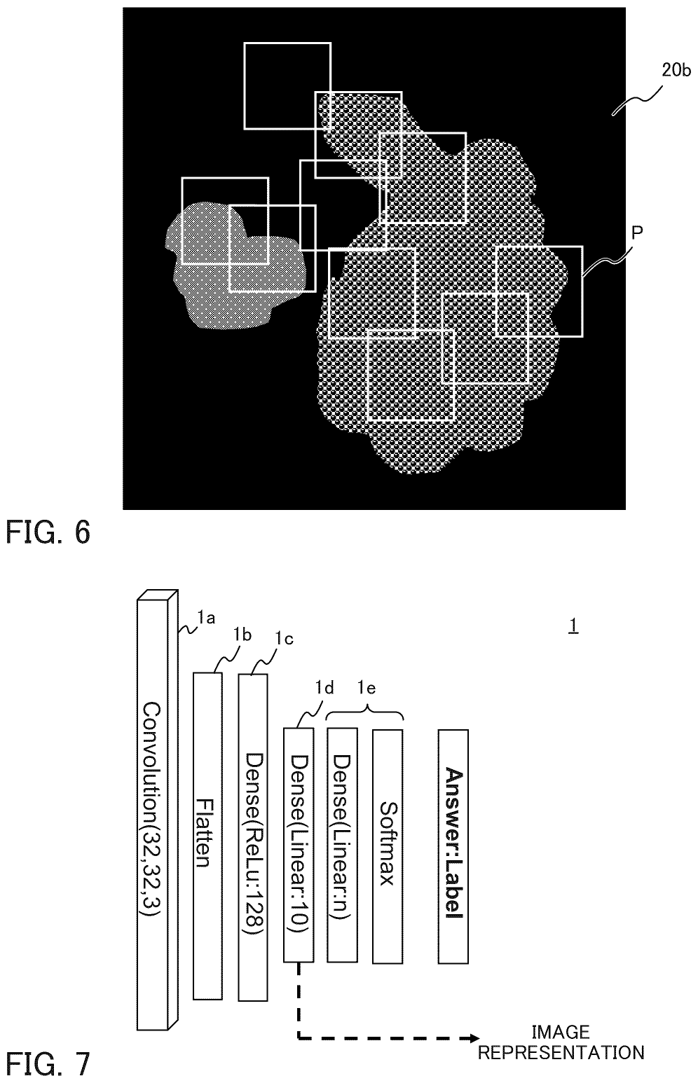

FIG. 6 is a diagram illustrating a state of generating image patches from a microscope image. FIG. 7 is a diagram illustrating a layer configuration example of the first model. With reference to FIG. 6 and FIG. 7, learning of the model 1 carried out by using collected microscope images will be described.

First, image patches of 64×64 pixels were generated from the collected microscope image (also referred to as original image), positions were randomly selected therefrom, and 100 patches were collected from the single original image. In this case, grayscale images were used. FIG. 6 illustrates the state of generating image patches P from the microscope image 20b. Each of the image patches P generated from the grayscale images was replicated three times to match the number of image patches generated from color images (color dimensions=3).

Then, learning to optimize the model was carried out by using the generated image patches P. Specifically, learning was carried out so as to minimize Sparse Categorical Cross-Entropy, which is a loss function, with respect to the input of the image patches P. This learning was carried out by randomly dividing the data set into subsets having a batch size of 100 every time. This was repeated for 100 epochs.

Note that the model 1 is a convolutional neural network (CNN), as illustrated in FIG. 7, includes a convolutional layer 1a, a flatten layer 1b subsequent thereto, and two layers, i.e., a dense layer 1c and a dense layer 1d, and further includes an output layer 1e which outputs the results finally processed by softmax function. The model 1 is designed so that the convolutional layer 1a outputs a vector quantity of 32×32×3 dimensions and the dense layer 1c and the dense layer 1d, which are intermediate layers, output vector quantities of 128 dimensions and 10 dimensions, respectively.

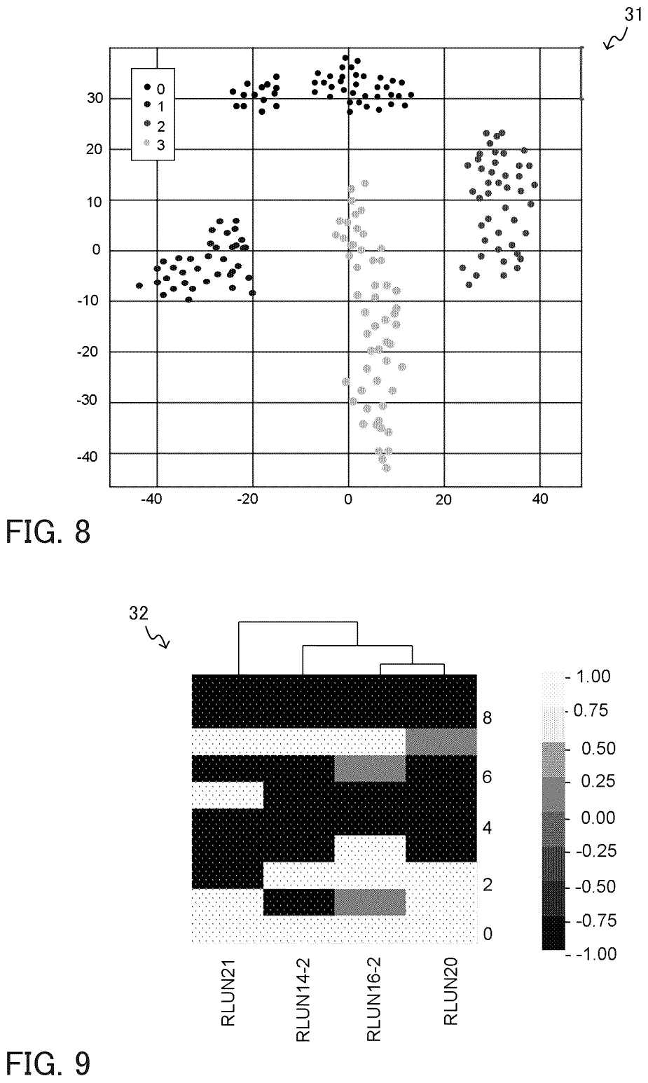

FIG. 8 is a diagram visualizing the image representations, which are obtained by the first model, by using a dimension reduction technique. FIG. 9 is a diagram visualizing patterns of the image representations. With reference to FIG. 8 and FIG. 9, the image representations 30 output from the intermediate layers of the model 1 will be described. Note that, in this case, the image representation 30 is a 10-dimensional vector quantity output from the dense layer 1d in the process of inferring the label from the microscope image.

FIG. 8 illustrates a state in which the image representation 30 is projected to low-dimensional space by t-distributed stochastic neighbor embedding (t-SNE). Each plot of a scatter diagram 31 illustrated in FIG. 8 corresponds to the image representation 30, which is obtained from one image patch, and is displayed by a different color depending on the label. As illustrated in FIG. 8, the plots corresponding to the image representations 30 obtained from the image patches to which the same label is attached are distributed to be mutually close, and the plots corresponding to the image representations 30 obtained from the image patches to which different labels are attached are distributed to be mutually separated. With reference to FIG. 8, it can be confirmed that the model 1 can output the image representation 30, which has lower dimensions than the image, as the information expressing a type of a cancer and from which patient it has been derived.

FIG. 9 is a heat map illustrating, by densities, the values obtained by averaging the image representations 30, which are obtained from the image patches to which the same label is attached, for each dimension. A vertical axis and a horizontal axis of the heat map 32 illustrated in FIG. 9 illustrate labels and element numbers of the image representations 30, respectively. More specifically, average values of the plurality of image representations 30 obtained from the plurality of image patches acquired from the images of the same organoid are illustrated as densities. With reference to FIG. 9, it can be confirmed that patterns of the image representations 30 are different, which show, for example, which dimension of the image representation 30 is intense and which dimension thereof is weak depending on differences in samples (labels).

As illustrated in FIG. 8 and FIG. 9, the image representations 30 are morphological representations identifiably expressing, by vector quantities of a plurality of dimensions, morphological differences between groups of cell clusters cultured from the same cancer specimen and groups of cell clusters cultured from other cancer specimens. Therefore, according to the appropriately learned model 1, the morphological features unique to the cancer of each of the patients expressed in images can be converted to and extracted as vector quantities (image representations 30) of a plurality of dimensions lower than those of the images.

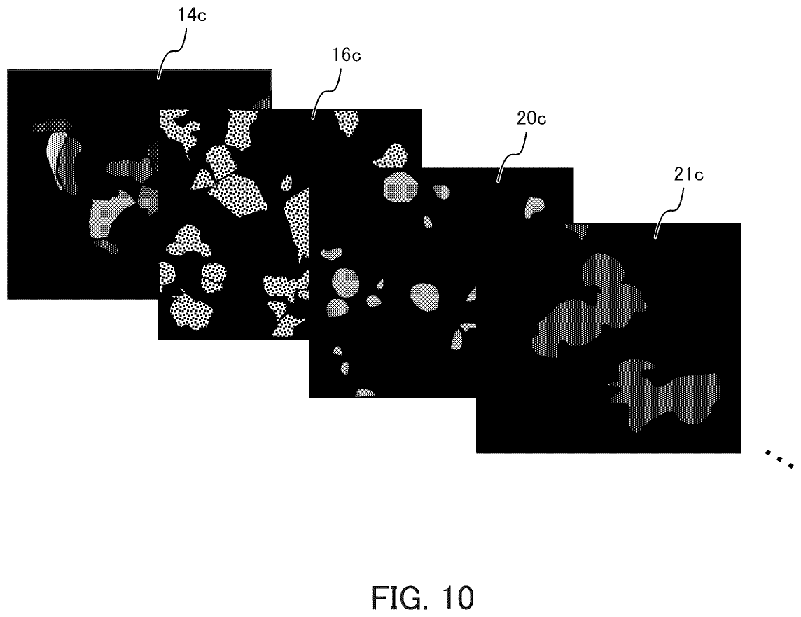

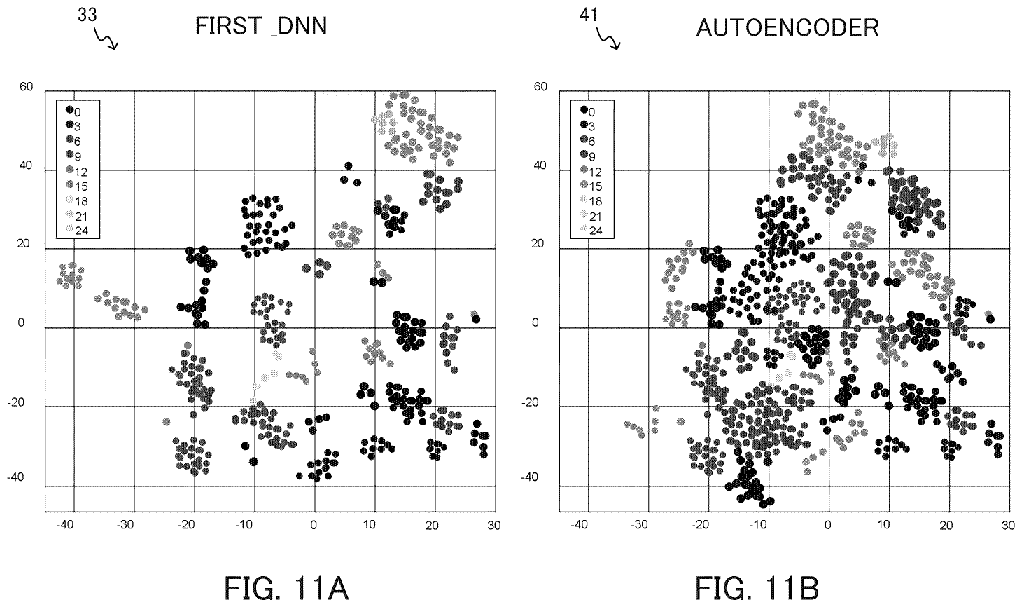

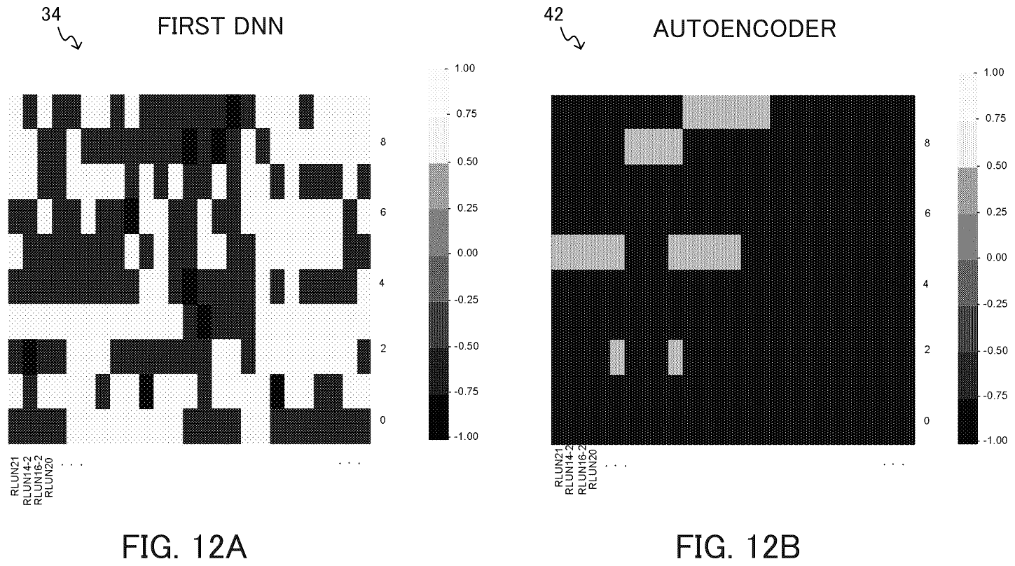

FIG. 10 is a diagram exemplifying color images acquired by color CCD. FIG. 11A and FIG. 11B are diagrams visualizing, by using the dimension reduction technique, the image representations 30 obtained by the first model and image representations obtained by an autoencoder, respectively. FIG. 12A and FIG. 12B are diagrams visualizing patterns of the image representations 30 obtained by the first model and patterns of the image representations obtained by the autoencoder, respectively. With reference to FIG. 10 to FIG. 12B, robustness and stability of the method of acquiring the image representation 30 by using the first model and requirements for the first model will be described.

The data set of the color images illustrated in FIG. 10 is the data set of the above described color images of 1920×1440 pixels acquired by using the 10-power bright-field objective lens and the color CCD and includes the image 14c, the image 20c, the image 16c, and the image 21c illustrated in FIG. 2C, FIG. 3C, FIG. 4C, and FIG. 5C. FIG. 8 and FIG. 9 illustrate the results of the case in which the grayscale images are used. However, the color images illustrated in FIG. 10 may be used to acquire the image representations 30. Also in this case, similar results can be obtained.

Specifically, as illustrated in a scatter diagram 33 of FIG. 11A, also in the case in which the color images are used, the model 1 can output the image representation 30 as the information expressing a type of a cancer and from which patient it has been derived. Also, as illustrated in a heat map 34 of FIG. 12A, also in the case in which the color images are used, with the model 1, it can be confirmed that the patterns of the image representations 30 are different depending on different samples (labels).

Therefore, according to the model 1, regardless of capture settings such as grayscale images or color images, the image representations 30 can be output as morphological representations identifiably expressing, by vector quantities of a plurality of dimensions, morphological differences between groups of cell clusters cultured from the same cancer specimen and groups of cell clusters cultured from other cancer specimens.

Instead of the model 1, another neural network model using an autoencoder (AE) was tested. In the AE model, learning is carried out so that the same images as input images are rebuilt. The AE model used in this case encoded input images by a layer assembly including a convolutional layer of 32×32×3, a dropout layer (dropout rate=0.1), and a max pooling layer of 2×2. Furthermore, this layer assembly was applied three times, and processing was further carried out with a flatten layer, a layer including 1024 nodes, and a layer including 10 nodes. Then, the encoded information was decoded with a similar configuration. As hyperparameters of learning, a batch size of 100 and the number of epochs of 100 were used.

The image representation obtained from the layer including 10 nodes, which is the intermediate layer of the above described AE model, is a vector quantity of a plurality of dimensions lower than those of the input images as well as the image representation 30 obtained from the model 1. However, as illustrated in a scatter diagram 41 of FIG. 11B, the image representations generated by the AE model are randomly distributed in output space by using t-SNE regardless of samples and do not express types of cancers and from which patients they are derived. Also, as illustrated in a heat map 42 of FIG. 12B, the image representations generated by the AE model have similar patterns regardless of samples (labels).

According to this result, it can be confirmed that the AE model cannot capture the features of individual cancer clusters, which are identified by labels, well. A reason therefor is that the AE model ignores labels of organoids and simply executes processing with respect only to images. Also in this case, the image representation of each patch can be obtained. However, as is understood with reference to FIG. 12A and FIG. 12B, compared with the image representations 30 obtained by the model 1, it can be confirmed that differences between original tissues (differences between labels) are reduced. According to this result, it can be understood that, in order to acquire the representations of organoids, a model like the model 1 which compares a plurality of image groups of the organoids having different labels and extracts common features in addition to the features of individual images is required.

Therefore, the AE model cannot be used instead of the model 1 to capture features of individual cancer clusters. The first model should be built like the model 1 so that the image representations are output as morphological representations identifiably expressing, by vector quantities of a plurality of dimensions, morphological differences between groups of cell clusters cultured from the same cancer specimen and groups of cell clusters cultured from other cancer specimens. In order to do this, for example, the first model is desired to be built as a classification model or a regression model which outputs information related to features of individual cancers such as labels.

In the above described example, the first model can be also described as the one that extracts the image representations expressing differences in labels of images (cancer tissues serving as deriving sources). However, the first model may be a regression model which identifies groups which are grouped by other indicators instead of the labels attached to the images. Also in this case, the image representations representing morphological features common to the organoids of the groups identified by the other indicators can be extracted. The other indicators are, for example, clinical data such as pathological diagnosis results and is not limited to clinical data per se, but may be the information which specifies the groups determined by doctors or the like by using the clinical data. In other words, the first model may be a model which acquires morphological representations so that morphological differences between a plurality of groups, which classify a plurality of cancer specimens, can be identified by using clinical data acquired in the process of pathological diagnosis.

Also, in the above described example, the image representations 30 are extracted from the single image. However, for example, an image of organoids after administering a medication may be acquired, and representations of morphological changes caused by administering the medication may be extracted by comparing the image with an image before administering the medication. This process further intensifies the differences between the labels. Therefore, improvement in prediction accuracy of gene expression levels in a later-described procedure can be expected. Moreover, improvement in prediction accuracy of medication reaction in a later-described procedure can be also expected.

<Second Model>

The model 2, which is a second model, predicts gene expression levels (prediction values 50) from the image representations 30. Hereinafter, in order to distinguish the gene expression levels predicted by the model 2 from the gene expression levels measured by measurement equipment such as a sequencer, the former will be described as prediction values of gene expression levels, and the latter will be described as measured values of gene expression levels in accordance with needs. The gene expression levels (prediction values 50), which are inference results of the model 2, are used to evaluate the prediction accuracy of each gene by comparing with the measured values. Furthermore, based on prediction accuracy of each gene, gene candidates related to cancers are extracted.

Generally, features related to morphologies and medication reaction of cancers and organoids are evaluated by a small number of genes. Therefore, in order to show the relevance between the genes and the microscope images, the correlations with gene expression, which is one of basic biological profiles of PDO, were analyzed by using the image representations 30 extracted by the first model.

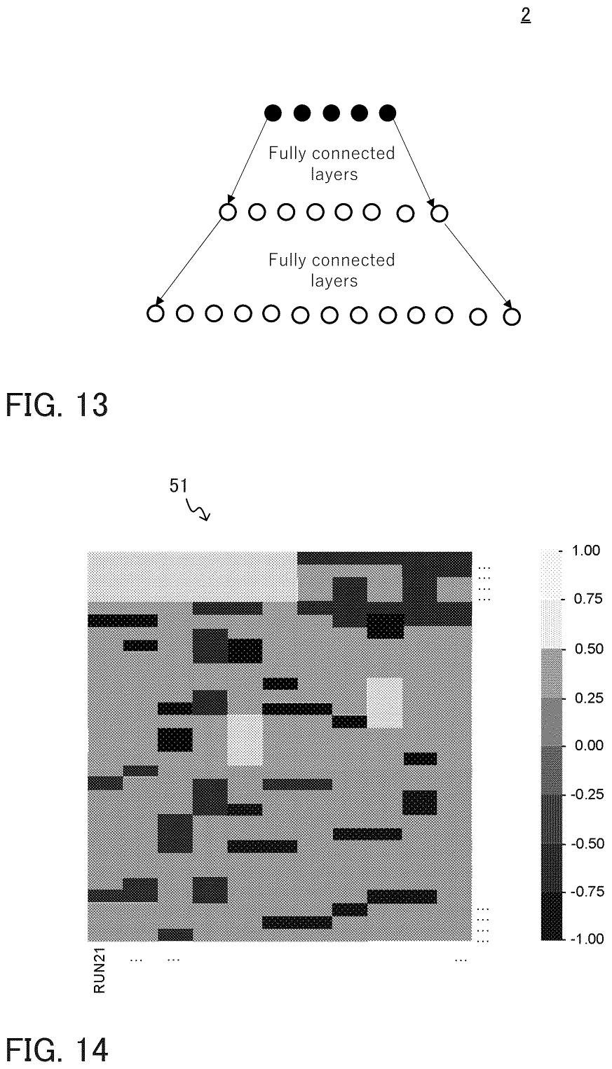

FIG. 13 is a diagram illustrating a layer configuration example of the second model. For the analysis, the model 2 which is a regression model of a deep neural network (DNN) as illustrated in FIG. 13 was learned. The model 2 is a model which uses, as input data, the 10-dimensional image representations 30 output from the model 1 and predicts the expression levels of 14400 genes. In the learning, 25 different samples were used. The gene expression level of each sample was measured, and the learning was carried out so that the output of the model 2 becomes close to the measured value of the gene expression level.

The learning carried out with respect to the model 2 can be also described as fitting of the model 2, which is a function, so that the gene expression levels measured from the samples are output with respect to input of the image representations 30 output from the model 1.

FIG. 13 is illustrated in a simplified manner, but the model 2 has fully connected layers including four layers having dimensions of 10, 18, 54, and 162, respectively, and has output dimensions of 14400. Each layer has fully linear connection not using an activating function. Mean squared error was used as a loss function, and learning was carried out to minimize the value thereof. As hyperparameters of the learning, a batch size of 50 and the number of epochs of 15 were used with respect to the number of input data N.

Generally, with respect to input data of images, effectiveness of Convolution processing that compares adjacent pixel information is known well. Therefore, the first model, which uses images as input data, uses the method of Convolutional Neural Network (CNN). However, since the second model does not use images as input, such processing is not necessarily required. Therefore, the second model employs a simple Deep Neural Network which does not execute Convolution processing (a neural network which combines a plurality of layers and is generally known as a deep learning technique).

The model 2 predicts gene expression levels based on the image representations 30. However, the input data of the model 2 is not limited to the image representations 30. Data which is a combination of the image representations 30 and other auxiliary data may be used as the input data. For example, “biochemical data such as cell activity” and “clinical data acquired in the process of diagnosis or treatment of patients” is widely used as indicators that feature tumors or patients. Connecting the data with the image representations 30 (data coupling (concatenating)) is generally widely used in neural network processing techniques, and the model 2 may use such data as input data.

FIG. 14 is a diagram visualizing gene expression levels measured for each label. The gene expression data set of FDO includes profiles estimated from the expression levels of 14400 human transcripts of each of 25 different samples (labels). A heat map 51 illustrated in FIG. 14 selects 100 genes among them in consideration of variance of gene expression levels between the samples and illustrates only the selected ones. In the heat map 51, a horizontal axis illustrates the labels, a vertical axis illustrates the genes, and the gene expression levels are illustrated by densities.

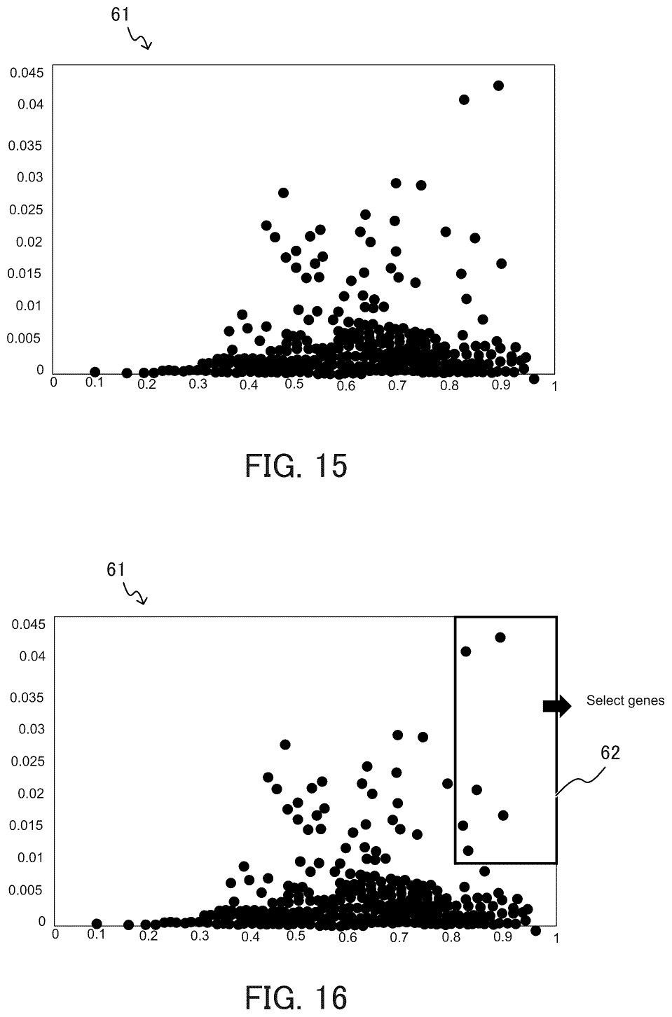

FIG. 15 and FIG. 16 are diagrams illustrating the relations between prediction accuracy and variance of the gene expression levels of the genes. The plots of scatter diagrams 61 illustrated in FIG. 15 and FIG. 16 correspond to the genes, respectively. The scatter diagram 61 is created for each sample (or each sample group of cancers of the same type). The vertical axis and the horizontal axis of the scatter diagram 61 illustrated in FIG. 15 and FIG. 16 illustrate the prediction accuracy of the gene expression levels and the variance of the gene expression levels, respectively, and are normalized by a range of [0,1]. The genes in the area 62 illustrated in FIG. 16 were extracted as gene candidates of the sample.

Specifically, first, the prediction values of the gene expression levels obtained by the model 2 by using the image representations 30 obtained by the model 1 as input were averaged with respect to each organoid sample to calculate a prediction value 50 of the gene expression level representing each gene. More specifically, the above described prediction was carried out with 25 samples by using a cross-validation method (3-fold cross-validation). Validation was carried out with respect to randomly selected three samples, and the model 2 was subjected to learning with respect to the data set of the remaining 22 samples. This learning was repeated 10 times, and the expression level of each gene was predicted for each sample by using the validation data set. After the prediction values 50 were calculated, the prediction accuracy thereof was evaluated. Ten prediction values 50 obtained by repeating the prediction ten times were used as one set, and the prediction accuracy thereof was evaluated by Pearson's Correlation Coefficient of the prediction values 50 and the answers (measured values). On the other hand, variance was calculated not from each sample, but from the measured values of 18 samples.

In the scatter diagrams 61 illustrated in FIG. 15 and FIG. 16, the genes corresponding to the plots in the area of high prediction accuracy in the comparatively right side mean that the expression levels of the genes can be predicted from the image representations of the organoids thereof. In other words, it is conceived that these genes are highly correlated to the image representations of the organoids thereof and are candidate genes which cause morphological differences. On the other hand, it is conceived that, in the scatter diagrams 61, the genes corresponding to the plots in the area of low prediction accuracy in the comparatively left side are not relevant to morphological diversity of PDO since the gene expression levels cannot be predicted from the image representations of the organoids thereof.

Therefore, the genes in the area in the comparatively right side in the scatter diagrams 61 can be considered as major gene candidates which exhibit the features of the cancers of individual patients. Furthermore, in order to improve gene selection accuracy, it is desired to use the variance, which represents statistical variation of expression levels, as an additional reference. A reason therefor is that small variations in the expression levels among different samples mean that these genes are common among the sample groups or are completely inactive. Therefore, the genes with small variance are excluded from the gene candidates related to morphological changes of PDO. The remaining genes are the genes plotted in the area 62 of FIG. 16 which are the genes with high variance and high correlation between prediction and measurement. It is highly possible that these genes have changed by reflecting the features of each sample, and the genes are major gene candidates exhibiting the features of the cancers of individual patients.

<Third Model>

The model 3, which is a third model, predicts drug response 70 from a set 60 of the gene candidates selected based on the prediction accuracy and variance calculated by using the model 2. In order to confirm the effect of selecting the gene candidates by using the model 2, regarding drug reaction which is another feature profile of PDO, prediction accuracy was estimated by using the gene candidates.

First, as illustrated in the area 62 of FIG. 16, the genes with high variance were specified, and the genes with high prediction accuracy were selected by a threshold value. In this case, the threshold value of the prediction accuracy is set to 0.8, which is a sufficiently large value as a correlation coefficient of the prediction value and an experimental value. Then, depending on an object, an arbitrary number n (n is the number of genes: n=3, 5, 8, 10) of genes were selected from the area 62 surrounded by the threshold value, and these genes were determined as gene candidates. The validity of the determined number n of genes can be validated, for example, by evaluating the prediction accuracy of drug responsiveness described later with reference to FIG. 19.

In this case, the n genes are selected in the descending order of variance. On the other hand, if this model is applied to another data set, the values of variance and the distribution thereof are different. In such a case, as a method, the value of n can be fixed, and, for example, the genes of n=10 can be selected in the descending order of the values of variance. Alternatively, the threshold value of variance can be fixed, and all the genes in the area 62 can be selected. Furthermore, this can be carried out by a plurality of methods. For example, the threshold values of the variance and the correlation coefficient can be arbitrarily changed by a user to select genes.

In this example, a gene candidate selecting method combining variance was executed. However, as a simpler case, gene candidates can be selected by using only the prediction accuracy as an indicator.

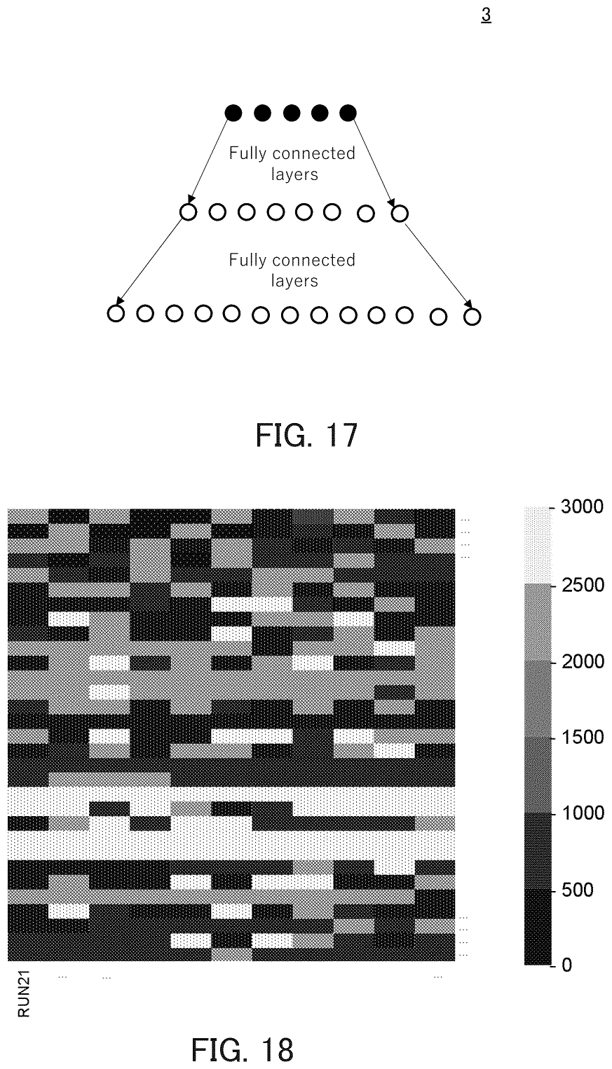

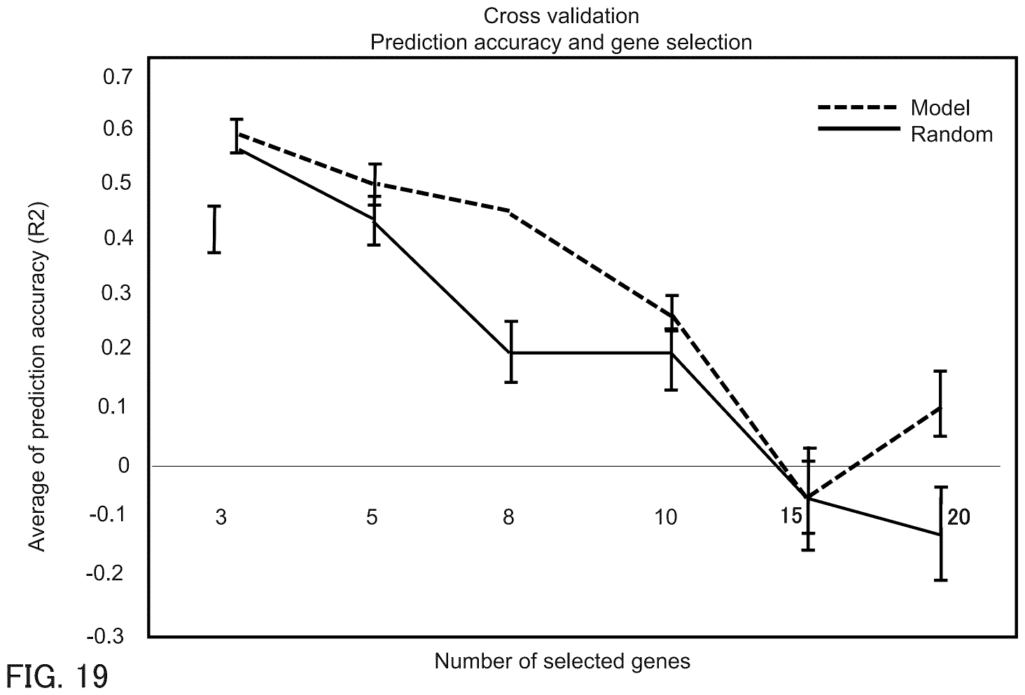

By using a set of the selected gene candidates, medication reaction was predicted by the model 3 illustrated in FIG. 17. FIG. 17 is a diagram illustrating a layer configuration example of the third model; FIG. 18 is a diagram visualizing the drug efficacy measured for each label. As illustrated in FIG. 17, the model 3 is a DNN regression model having fully connected layers including three layers. Mean squared error is used as a loss function. Learning was carried out to minimize the mean squared error of the output values of the model 3 illustrated in FIG. 17 and the measured values illustrated in FIG. 18. Drug response evaluated by AUC values regarding 76 chemical substances for each of 18 samples was predicted by using the regression model. The prediction accuracy was evaluated by using 18 samples by a cross-validation method (five-fold cross-validation).

FIG. 19 is a diagram illustrating prediction accuracy of drug responsiveness. As illustrated in FIG. 19, in the results around three genes and five genes, the value of a determination coefficient R2 (A statistical indicator generally used to evaluate prediction accuracy. This is given by a definition similar to Pearson's Correlation Coefficient) is about 0.5, and it can be understood that the performance of the neural network model is moderate. Also, according to FIG. 19, it can be understood that the above described gene candidate selection model using the model 2 maintains comparatively high prediction accuracy from three genes to ten genes compared with a case of random selection. These results show that the gene candidate selection model is effective for medication reaction prediction. It can be evaluated that it is appropriate to select, as the number n of genes, a value of about three to ten, with which higher prediction accuracy than random selection can be obtained.

In this case, the medication reaction prediction was carried out based on the selected gene candidates, but other relevant data can be also applied to prediction by a similar method. In fact, classifying patient groups (stratification of patients) or carrying out detailed diagnosis according to the data of a plurality of gene groups has been widely carried out.

<System Configuration Example>

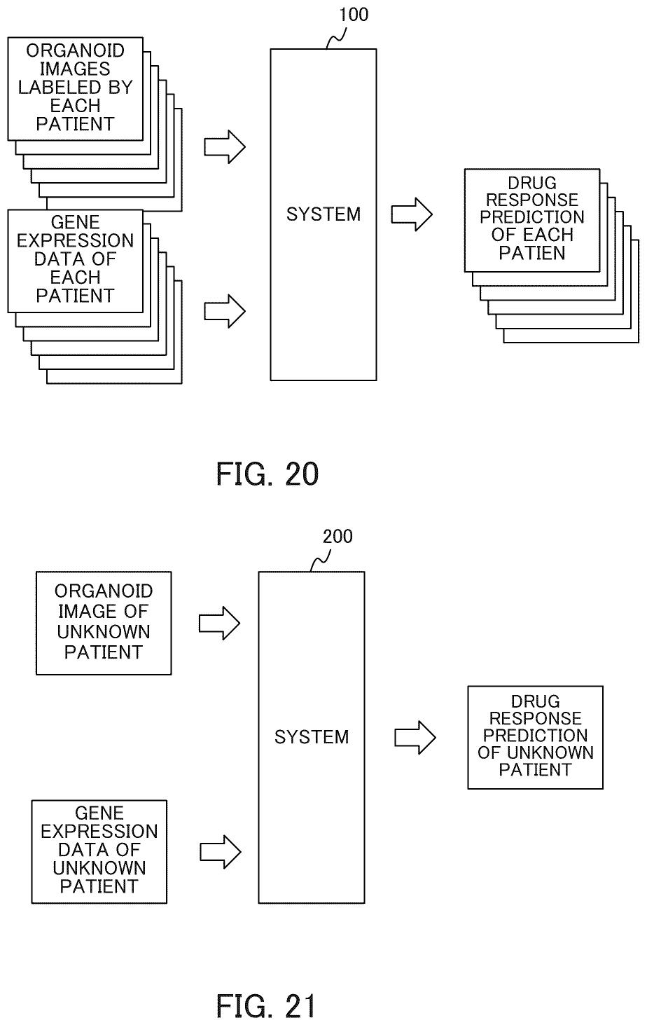

FIG. 20 and FIG. 21 are diagrams illustrating system configuration examples using the above described models. A system 100 illustrated in FIG. 20 is a system including the model 1 and the model 2 described above. In the system 100, as illustrated in FIG. 20, the model 1 is subjected to learning by using a plurality of organoid images labeled by each patient, and the model 2 is subjected to learning by using the output (image representations) from the model 1 and the gene expression data of the plurality of patients. As a result, by setting threshold values of the prediction accuracy, variance, etc., the gene candidates related to the cancers of a plurality of respective patients can be provided to a user. Note that the method of providing the gene candidates is not particularly limited. For example, the gene candidates can be provided to the user by displaying the gene candidates on a display device. Also, the gene candidates may be stored in a storage device so that the gene candidates can be read at required timing. Other than that, the gene candidates may be provided to the user by printing, e-mails, etc.

If the learning of the model 1 has been completed, the images input to the system 100 are not necessarily required to be labeled. Organoid images of a plurality of patients and gene expression data of the plurality of patients may be input. Also in this case, gene candidates can be extracted for each of the patients.

A system 200 illustrated in FIG. 21 is a system including the model 1, the model 2, and the model 3 described above. In the system 200, the learning of the model 1, the model 2, and the model 3 is carried out in advance. In this case, only by inputting an organoid image of an unknown patient together with the gene expression data of the patient, medication exhibiting high medication reaction can be specified from the gene candidates related to the cancer of the patient. Therefore, the medication that is effective to treatment, etc. of the patient can be output together with the degree of the effect. An example of the output information is (medication A: effectiveness 1.0, medication B: effectiveness 0.6, medication C: effectiveness 0.1). In this manner, not only effective medication, but also medication with low effectiveness can be also predicted.

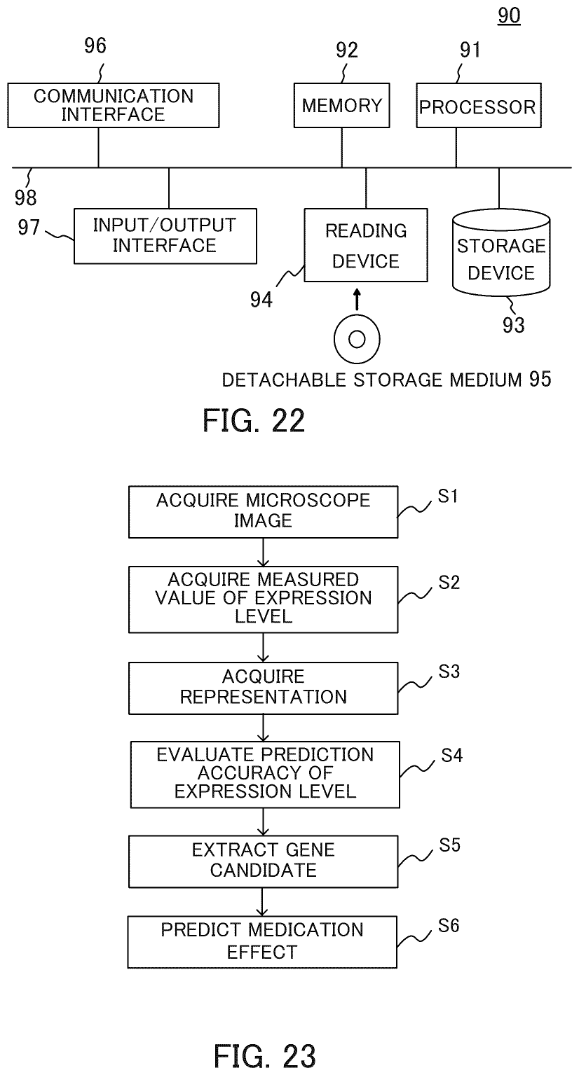

FIG. 22 is a diagram exemplifying a hardware configuration of a computer 90 for implementing the above described system. The hardware configuration illustrated in FIG. 22 includes, for example, a processor 91, a memory 92, a storage device 93, a reading device 94, a communication interface 96, and an input/output interface 97. The processor 91, the memory 92, the storage device 93, the reading device 94, the communication interface 96, and the input/output interface 97 are mutually connected, for example, via a bus 98.

The processor 91 reads out a program stored in the storage device 93 and executes the program, thereby operating the above described model. For example, the memory 92 is a semiconductor memory, and may include a RAM area and a ROM area. The storage device 93 is, for example, a hard disk, a semiconductor memory such as a flash memory, or an external storage device.

For example, the reading device 94 accesses a storage medium 95 in accordance with an instruction from the processor 91. For example, the storage medium 95 is implemented by a semiconductor device, a medium to/from which information is input/output by magnetic action, or a medium to/from which information is input/output by optical action.

For example, the communication interface 96 communicates with other devices in accordance with instructions from the processor 91. The input/output interface 97 is, for example, an interface between an input device and an output device. For example, a display, a keyboard, a mouse, etc. are connected to the input/output interface 97.

For example, the program executed by the processor 91 is provided to the computer 90 in the following forms:
(1) Installed in the storage device 93 in advance,
(2) Provided by the storage medium 95, and
(3) Provided from a server such as a program server.

Note that the hardware configuration of the computer 90 for implementing the system described with reference to FIG. 22 is exemplary, and the embodiment is not limited thereto. For example, part of the configuration described above may be omitted or a new configuration may be added to the configuration described above. In another embodiment, for example, some or all of the functions of the electric circuit described above may be implemented as hardware based on a field programmable gate array (FPGA), a system-on-a-chip (SoC), an application specific integrated circuit (ASIC) or a programmable logic device (PLD).

The above-described embodiments are specific examples to facilitate an understanding of the invention, and hence the present invention is not limited to such embodiments. Modifications obtained by modifying the above-described embodiments and alternatives to the above-described embodiments may also be included. In other words, the constituent elements of each embodiment can be modified without departing from the spirit and scope of the embodiment. Moreover, new embodiments can be implemented by appropriately combining a plurality of constituent elements disclosed in one or more of the embodiments. Furthermore, some constituent elements may be omitted from the constituent elements in each of the embodiments, or some constituent elements may be added to the constituent elements in each of the embodiments. Moreover, the order of the processing procedure described in each of the embodiments may be changed as long as there is no contradiction. That is, the method of extracting gene candidates, the method of utilizing gene candidates, and the computer-readable medium according to the present invention can be variously modified or altered without departing from the scope of the claims.

For example, deep learning techniques are not necessarily required to be used in the above described three models. For example, as long as the representations unique to the cancers of patients can be extracted, the first model, which extracts image representations, may extract the image representations designed by human in advance instead of CNN, for example, may extract the sizes and morphological degrees (for example, rounded unevenness or the like) of organoid areas in images identified by outline shapes, etc. as image representations. Also, the second model, which outputs gene expression levels, may replace the neural network with a function obtained by using a general regression analysis method (for example, the least squares method, which is the simplest one) for fitting with respect to measured gene expression levels. The same as the second model applies also to the third model. Note that, in all of the first to third models, if target data is complex, general deep learning techniques are effective. However, comparatively simple cases (the cases in which the number of sample groups is smaller than this case or only a smaller number of gene groups are used as input) are not limited to deep learning techniques, but can use comparatively simple methods as described above.

FIG. 23 is a flow chart of a process of extracting and utilizing the above described gene candidates related to the features of cancers of individual patients. As illustrated in FIG. 23, the method of extracting the gene candidates is desired to include the following six procedures.

1. A procedure of acquiring a microscope image of a cultured cell cluster derived from a cancer specimen of a patient (step S1)

2. A procedure of acquiring a measured value of a gene expression level of the cancer specimen used in the procedure 1. or the cell cluster cultured from the cancer specimen (step S2)

3. A procedure of, based on the microscope image acquired in procedure 1., acquiring a morphological representation identifiably expressing, by an identifiable vector quantity of a plurality of dimensions, morphological differences between a group of cell clusters cultured from the same cancer specimen and a group of cell clusters cultured from another cancer specimen (step S3)

4. A procedure of inputting the morphological representation, which has been acquired in the procedure 3., to the function, which has been obtained by fitting using the morphological representation as input and the measured value of the gene expression level as output, thereby estimating the prediction accuracy of the gene expression level based on the acquired prediction value of the gene expression level and the measured value of the gene expression level acquired in the procedure 2. (step S4)

5. A procedure of extracting the genes related to morphological changes of the cell clusters as gene candidates based on the prediction accuracy estimated in the procedure 4. (step S5)

6. A procedure of supporting classification or diagnosis of the cancer of the patient or predicting effects of medication with respect to the patient based on the gene candidates extracted in the procedure 4. (step S6)

In step S1, the microscope image may be acquired by using a microscope, or an already acquired microscope image may be acquired. In step S1, the microscope image of the cell cluster may be acquired before administering medication to the cell cluster, and another microscope image of the cell cluster may be acquired after administering the medication to the cell cluster. The changes in these images may be used as the input of step S3.

In step S2, the measured value of the gene expression level of the sample (cancer specimen, cell cluster) related to the microscope image acquired in step S1 is acquired. Herein, the measurement may be carried out by using a sequencer or the like, or an already-measured measured value may be acquired.

In step S3, the morphological representation is acquired based on the microscope image acquired in step S1. Herein, the morphological representation (image representation) can be acquired by using the first model described above. Note that, if the first model has already been learned, the microscope image is not required to be labeled. In this case, the morphological representation may be acquired by using deep learning techniques.

Note that the morphological representation is a representation with which morphological differences between a plurality of groups, which classifies a plurality of cancer specimens by using clinical data acquired in the process of pathological diagnosis, can be identified. In step S3, such morphological representation is acquired.

In step S4, the gene expression level is predicted based on the morphological representation acquired in step S3, and the prediction accuracy of each gene is estimated from comparison with the measured value. The above described second model can be used to predict the gene expression level.

Note that, before step S4, a procedure of fitting the function which outputs the measured value of the gene expression level acquired in step S2 with respect to the input of the morphological representation acquired in step S3 may be provided. The second model may be optimized by this procedure. In this case, the fitting of the function may be carried out by using deep learning techniques.

Furthermore, as the input used for fitting of the function, in addition to the morphological representation, biochemical data other than the gene expression level of the cancer specimen or the cell cluster cultured from the cancer specimen may be used, or clinical data acquired in the process of diagnosis or treatment of the patient may be used. Therefore, a procedure of acquiring the data may be provided before the fitting procedure. In such a case, it is desired that the combination of the morphological representation and the data be input to the function also in step S4.

In step S5, the gene candidates are extracted based on the prediction accuracy of each gene estimated in step S4. Specifically, the genes with high prediction accuracy can be preferentially extracted. Further desirably, the genes with high prediction accuracy and large variance of expression levels between samples are preferentially extracted. More specifically, step S5 may include a procedure of statistically estimating variation in the measured values of the gene expression levels and a procedure of extracting the gene candidates based on the magnitude of the estimated variation and the prediction accuracy estimated in step S4. Note that the extracted gene candidates may be displayed on a display device or may be output to a file.

In step S6, according to the gene candidates extracted in step S5, diagnosis of judging the type of the cancer of the patient is supported. Alternatively, according to the gene candidates extracted in step S5, the effects of each medication on the patient are predicted. The third model can be used to predict the effects of the medication.

What is claimed is:

1. A method of extracting a gene candidate related to a feature of a cancer of an individual patient, the method comprising:
(a) acquiring a microscope image of a cultured cell cluster derived from a cancer specimen of the patient;
(b) acquiring a measured value of a gene expression level of the cancer specimen or the cell cluster cultured from the cancer specimen used in the (a);
(c) acquiring a morphological representation identifiably expressing, by a vector quantity of a plurality of dimensions, a morphological difference between a group of a cell cluster cultured from the same cancer specimen and a group of a cell cluster cultured from another cancer specimen based on the microscope image acquired in the (a);
(d) estimating prediction accuracy of the gene expression level based on a prediction value of the gene expression level and the measured value of the gene expression level acquired in the (b), the prediction value being acquired by inputting the morphological representation acquired in the (c) to a function obtained by fitting using the morphological representation as input and the measured value of the gene expression level as output; and
(e) extracting a gene related to a morphological change of the cell cluster as the gene candidate based on the prediction accuracy estimated in the (d).

2. The method according to claim 1, wherein
the (a) includes
acquiring the microscope image of the cell cluster before administering medication to the cell cluster, and
acquiring the microscope image of the cell cluster after administering the medication to the cell cluster.

3. The method according to claim 1, further comprising
(f) fitting the function that outputs the measured value of the gene expression level acquired in the (b) with respect to the input of the morphological representation acquired in the (c).

4. The method according to claim 2, further comprising
(g) acquiring biochemical data of the cancer specimen or the cell cluster cultured from the cancer specimen used in the (a), the biochemical data being other than the gene expression level, or acquiring clinical data acquired in process of diagnosis or treatment of the patient, wherein,
in the (f), the function is subjected to fitting so that the measured value of the gene expression level acquired in the (b) is output with respect to the input of a combination of the data acquired in the (g) and the morphological representation acquired in the (c).

5. The method according to claim 1, wherein
in the (c), the morphological representation identifiably expressing a morphological difference between a plurality of groups classifying a plurality of cancer specimens by using clinical data acquired in process of pathological diagnosis is acquired.

6. The method according to claim 1, wherein
the acquiring the morphological representation in the (c) is carried out by using a deep learning technique.

7. The method according to claim 1, wherein
the fitting of the function in the (f) is carried out by using a deep learning technique.

8. The method according to claim 1, wherein
the (e) includes
statistically estimating variation in the measured value of the gene expression level, and
extracting the gene candidate based on the prediction accuracy and magnitude of the variation.

9. A method of utilizing a gene candidate extracted by using the method of extracting the gene candidate according to claim 1, the method comprising
supporting classification or diagnosis of a cancer of a patient or predicting an effect of medication with respect to the patient based on the extracted gene candidate.

10. A non-transitory computer-readable medium storing a program that causes
a computer to execute:
(a) acquiring a microscope image of a cultured cell cluster derived from a cancer specimen of a patient;
(b) acquiring a measured value of a gene expression level of the cancer specimen or the cell cluster cultured from the cancer specimen used in the (a);
(c) acquiring a morphological representation identifiably expressing, by a vector quantity of a plurality of dimensions, a morphological difference between a group of a cell cluster cultured from the same cancer specimen and a group of a cell cluster cultured from another cancer specimen based on the microscope image acquired in the (a);
(d) estimating prediction accuracy of the gene expression level based on a prediction value of the gene expression level and the measured value of the gene expression level acquired in the (b), the prediction value being acquired by inputting the morphological representation acquired in the (c) to a function obtained by fitting using the morphological representation as input and the measured value of the gene expression level as output; and
(e) extracting a gene related to a morphological change of the cell cluster as the gene candidate related to a feature of a cancer of the patient based on the prediction accuracy estimated in the (d).
